(19) United States
(12) Patent Application Publication
(10) Pub. No.: US 2025/0375566 A1
(43) Pub. Date: Dec. 11, 2025

(54) FLOW INTERFACE APPARATUS USABLE WITH WOUND IRRIGATION DEVICE AND METHODS OF USE THEREOF

(71) Applicant: CAREFUSION 2200, INC., San Diego, CA (US)

(72) Inventors: Karan KERWELL, San Diego, CA (US); Christopher MCGINLEY, San Diego, CA (US)

(21) Appl. No.: 18/876,108

(22) PCT Filed: Jun. 16, 2023
(86) PCT No.: PCT/US2023/025545

§ 371 Date: Dec. 17, 2024

Related U.S. Application Data
(60) Provisional application No. 63/366,588, filed on Jun. 17, 2022.

(51) Int. Cl. A61M 3/02

(52) U.S. Cl. CPC A61M 3/0262

(57) ABSTRACT

An irrigation system, method, and interface apparatus. The interface apparatus includes a receiving portion configured to provide a sealed connection with a container interface of a solution container, wherein the receiving portion further comprises a spike interface therein. The spike interface includes a first fluid path for providing fluid communication between the solution container and the irrigation apparatus via the interface apparatus.

### CROSS REFERENCE TO RELATED APPLICATIONS

The present application claims priority to U.S. Provisional Application No. 63/366,588 entitled “FLOW INTERFACE APPARATUS USABLE WITH WOUND IRRIGATION DEVICE AND METHODS OF USE THEREOF,” filed Jun. 17, 2022, which is hereby incorporated by reference in the entirety.

### INTRODUCTION

The present disclosure generally relates to devices for administering wound irrigation, particularly a flow interface apparatus and wound irrigation device that employs a flow interface apparatus.

### BACKGROUND

Irrigation is an accepted method to aid in the prevention of surgical site infections (SSIs) and/or to otherwise provide flushing or dilution in patient procedures. For open procedures, irrigation is typically conducted in one of two ways: manually (e.g., directly pouring an irrigation solution, applying an irrigation solution via a bulb-type syringe or device, or applying an irrigation solution by manually squeezing an irrigation device); or via pulsatile lavage (e.g., electric pumping of an irrigation solution with increased pressure and flow rate) or gravity-based irrigation (e.g., the solution is provided from a container that is held or mounted higher than the irrigation site). Irrigation may also be applicable to open and/or closed procedures used in cystoscopy, Transurethral procedures (e.g., T.U.R), general, and arthroscopic surgical procedures. The current disclosure describes aspects and improvements that provide increased versatility, safety, and efficiency of a irrigation, flush, and/or lavage system (which may be pump operated or a gravity system), and for providing overall improved efficiency and safety of irrigation and/or flush systems, apparatuses, and methods.

### SUMMARY

The following presents a simplified summary of one or more aspects of the invention in order to provide a basic understanding of such aspects. This summary is not an extensive overview of all contemplated aspects, and is intended to neither identify key or critical elements of all aspects nor delineate the scope of any or all aspects. Its purpose is to present some concepts of one or more aspects in a simplified form as a prelude to the more detailed description that is presented later.

In some aspects, the techniques described herein relate to an interface apparatus usable with a solution container and an irrigation (e.g. lavage apparatus) and/or a flush system, the interface apparatus including: a receiving portion configured to provide a sealed connection with a container interface of the solution container, wherein the receiving portion further includes a spike interface therein; and the spike interface includes a first fluid path for providing fluid communication between the solution container and the lavage apparatus via the interface apparatus.

In some aspects, the techniques described herein relate to an interface apparatus, wherein the spike interface has a tip configured to pierce a portion of the solution container, wherein the first fluid path is configured to fluidly communicate with the solution container when the solution container is pierced.

In some aspects, the techniques described herein relate to an interface apparatus, wherein the spike interface includes a second fluid path, wherein the second fluid (e.g., air) path is configured to provide the solution container with fluid communication to an atmosphere outside the solution container via a vent when the solution container is pierced by the spike interface.

In some aspects, the techniques described herein relate to an interface apparatus, wherein the vent further includes at least one of a one-way valve, a membrane, or a sterile filter.

In some aspects, the techniques described herein relate to an interface apparatus, wherein the interface apparatus is configured to be installed onto the container interface of the solution container.

In some aspects, the techniques described herein relate to an interface apparatus, wherein the receiving portion further includes engagement threads that are configured to threadingly engage with threaded container engagement provisions of the container interface, wherein the threading engagement between the engagement threads and the threaded container engagement provisions causes the spike interface to pierce the solution container at the container interface as the interface apparatus is rotated with respect to the solution container.

In some aspects, the techniques described herein relate to an interface apparatus, further including a lavage port configured interface with and provide fluid communication from the solution container to a lavage apparatus connector.

In some aspects, the techniques described herein relate to an interface apparatus, wherein the lavage apparatus connector is a lavage spike, and wherein the lavage apparatus connector is configured to provide the fluid communication from the solution container to the lavage apparatus connector and lavage apparatus when the lavage spike is pressed into the lavage port.

In some aspects, the techniques described herein relate to an interface apparatus, wherein the lavage port further includes a lavage port seal, wherein the lavage port seal is configured to be pierced by the lavage spike when the lavage spike is pressed into the lavage port.

In some aspects, the techniques described herein relate to a lavage device and interface device, the lavage device including a nozzle and a pump in fluid communication with the nozzle, wherein the pump provides a pressurized stream of lavage solution supplied from a solution container, wherein the interface device includes: a receiving portion configured to provide a sealed connection with a container interface of the solution container, wherein the receiving portion further includes a spike interface therein; and the spike interface includes a first fluid path for providing fluid communication between the solution container and the lavage device via the interface apparatus.

In some aspects, the techniques described herein relate to a lavage device and interface device, wherein the spike interface has a tip configured to pierce a portion of the solution container, wherein the first fluid path is configured to fluidly communicate with the solution container when the solution container is pierced.

In some aspects, the techniques described herein relate to a lavage device and interface device, wherein the spike interface includes a second fluid path, wherein the second fluid path is configured to provide the solution container with fluid communication to an atmosphere outside the solution container via a vent when the solution container is pierced by the spike interface.

In some aspects, the techniques described herein relate to a lavage device and interface device, wherein the vent further includes at least one of a one-way valve, a membrane, or a sterile filter.

In some aspects, the techniques described herein relate to a lavage device and interface device, wherein the interface apparatus is configured to be installed onto the container interface of the solution container.

In some aspects, the techniques described herein relate to a lavage device and interface device, wherein the receiving portion further includes engagement threads that are configured to threadingly engage with threaded container engagement provisions of the container interface, wherein the threading engagement between the engagement threads and the threaded container engagement provisions causes the spike interface to pierce the solution container at the container interface as the interface apparatus is rotated with respect to the solution container.

In some aspects, the techniques described herein relate to a lavage device and interface device, further including a lavage port configured interface with and provide fluid communication from the solution container to a lavage apparatus connector.

In some aspects, the techniques described herein relate to a lavage device and interface device, wherein the lavage apparatus connector is a lavage spike, and wherein the lavage apparatus connector is configured to provide the fluid communication from the solution container to the lavage apparatus connector and lavage apparatus when the lavage spike is pressed into the lavage port.

In some aspects, the techniques described herein relate to a lavage device and interface device, wherein the lavage port further includes a lavage port seal, wherein the lavage port seal is configured to be pierced by the lavage spike when the lavage spike is pressed into the lavage port.

In some aspects, the techniques described herein relate to a method for providing a fluid path from a solution container to a lavage device, the method including: connecting a receiving portion of an interface apparatus to the solution container by puncturing a portion of the solution container with a spike interface of the interface apparatus, wherein puncturing a portion of the solution container with the spike interface establishes a fluid connection between the solution container and the lavage apparatus via the interface apparatus.

In some aspects, the techniques described herein relate to a method, wherein the receiving portion further includes engagement threads and the solution container further includes engagement provisions, and wherein the method further includes threadingly engaging the engagement threads of the receiving portion with the threaded container engagement provisions to provide a sealed interface between the interface apparatus and the solution container.

These and other aspects of the invention will become more fully understood upon a review of the detailed description, which follows.

### BRIEF DESCRIPTION OF THE DRAWINGS

FIG. 1 is a side view of an example interface apparatus and solution container according to aspects of the disclosure.

FIG. 2A is a side view of the example interface apparatus of FIG. 1 installed onto the solution container and connected to a lavage device according to aspects of the disclosure.

FIG. 2B-2D are side views of installation steps of an example interface apparatus installed onto a solution container and connected to a lavage device according to aspects of the disclosure.

FIG. 2E is a diagram view of multiple example interface apparatuses according to aspects of the disclosure.

FIG. 3 is a partial side view of the interface apparatus of FIGS. 1 and 2 according to aspects of the disclosure.

FIG. 4 is a cross-section view of the interface apparatus of FIGS. 1-3 according to aspects of the disclosure.

FIG. 5 is a cross-section view of the interface apparatus of FIGS. 1-4 showing example fluid paths according to aspects of the disclosure.

FIG. 6 is a cross-section view of the interface apparatus of FIGS. 1-6 showing an example lavage device interface according to aspects of the disclosure.

FIG. 7 is a perspective view of an interface apparatus according to aspects of the disclosure.

FIG. 8 is a cross-section view of the interface apparatus of FIG. 7 according to aspects of the disclosure.

FIG. 9 is a cross-section view of the interface apparatus of FIGS. 7 and 8 showing example fluid paths according to aspects of the disclosure.

FIG. 10 is a cross-section view of the interface apparatus of FIGS. 7-9 showing an example lavage device interface according to aspects of the disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known components are shown in simplified form in order to avoid obscuring such concepts.

### I. Terminology

Throughout the disclosure, the terms substantially or approximately may be used as a modifier for a geometric relationship between elements or for the shape of an element or component. While the terms substantially or approximately are not limited to a specific variation and may cover any variation that is understood by one of ordinary skill in the art to be an acceptable variation, some examples are provided as follows. In one example, the term substantially or approximately may include a variation of less than 10% of the dimension of the object or component. In another example, the term substantially or approximately may include a variation of less than 5% of the object or component. If the term substantially or approximately is used to define the angular relationship of one element to another element, one non-limiting example of the term substantially or approximately may include a variation of 5 degrees or less. These examples are not intended to be limiting and may be increased or decreased based on the understanding of acceptable limits to one of skill in the relevant art.

For purposes of the disclosure, directional terms are expressed generally with relation to a standard frame of reference when the system and apparatus described herein is installed in an in-use orientation. Further, in order to provide context to the current disclosure, a broad overview of the discovered deficiencies of various systems and an example implementation of the current disclosure and the advantages provided by the disclosure are described below. Further details of example implementations of the current disclosure are described in detail with reference to the figures below.

Terms such as a, an, and the are not intended to refer to only a singular entity, but include the general class of which a specific example may be used for illustration. The terms a, an, and the may be used interchangeably with the term at least one. The phrases at least one of and comprises at least one of followed by a list refers to any one of the items in the list and any combination of two or more items in the list.

The terms first, second, third, and fourth, among other numeric values, may be used in this disclosure. It will be understood that, unless otherwise noted, those terms are used in their relative sense only. In particular, in some aspects certain components may be present in interchangeable and/or identical multiples (e.g., pairs). For these components, the designation of first, second, third, and/or fourth may be applied to the components merely as a matter of convenience in the description of one or more of the aspects of the disclosure.

The term lavage as used throughout the disclosure is may include but is not limited to pulsed lavage, pulsatile jet lavage, or lavage. The term lavage however is not intended to be limited to a specific type of irrigation or flushing mechanism or system. For example, the term lavage may include flushing or irrigation systems and apparatuses typically used in open and/or closed procedures used in cystoscopy, transurethral procedures (e.g., T.U.R), general, and arthroscopic surgical procedures, to name a few non-limiting examples. Thus, the terms Lavage, irrigation, and/or flushing may be used interchangeably throughout the disclosure. In one example, the term lavage may include a device, system, or method that provides either a constant stream or pulsed stream of solution to irrigate and debride wounds of contaminants and/or necrotic tissue and/or for flushing or diluting bodily fluids or matter.

### II. Overview

For context, this overview is provided of aspects of the disclosure and the advantages the disclosure provides. This overview, and the detailed description that follows, has been presented for purposes of illustration and description. It is not intended to be exhaustive nor to limit the disclosure to the forms described. Numerous modifications are possible in light of the above teachings, including a combination of the abovementioned aspects. Some of those modifications have been discussed and others will be understood by those skilled in the art. The various aspects were chosen and described in order to best illustrate the principles of the present disclosure and various aspects as are suited to the particular use contemplated. The scope of the present disclosure is, of course, not limited to the examples or aspects set forth herein, but can be employed in any number of applications and equivalent devices by those of ordinary skill in the art. Rather, it is hereby intended the scope be defined by the claims appended hereto.

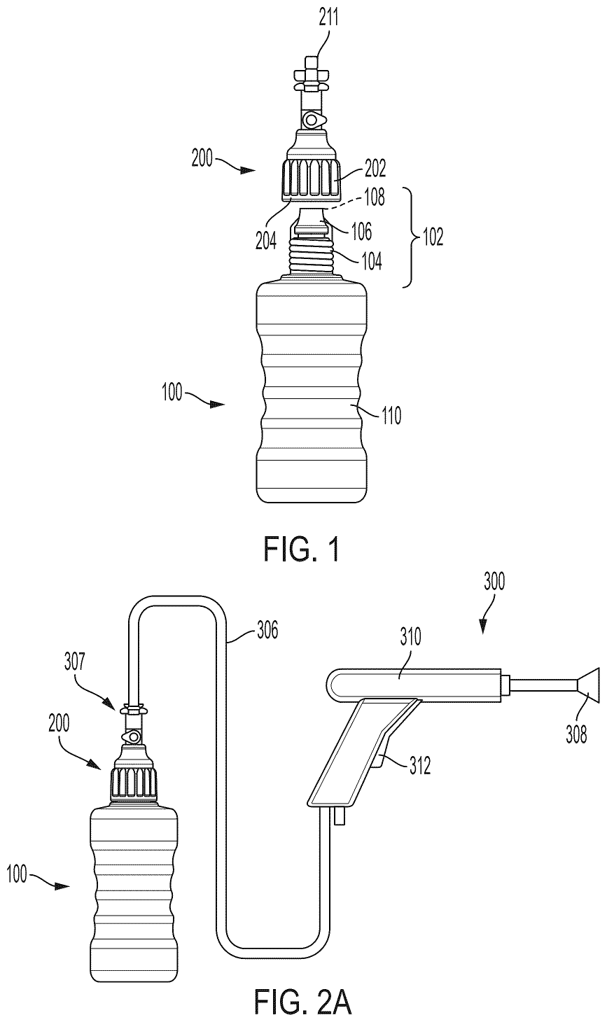

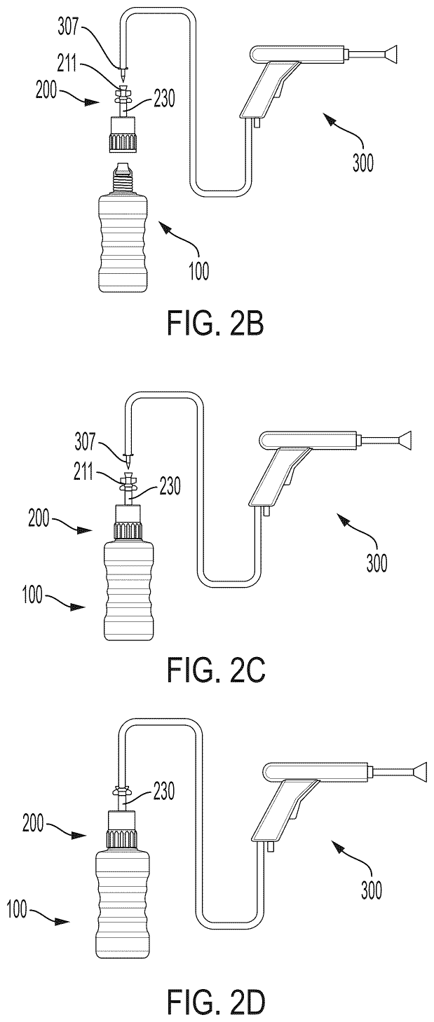

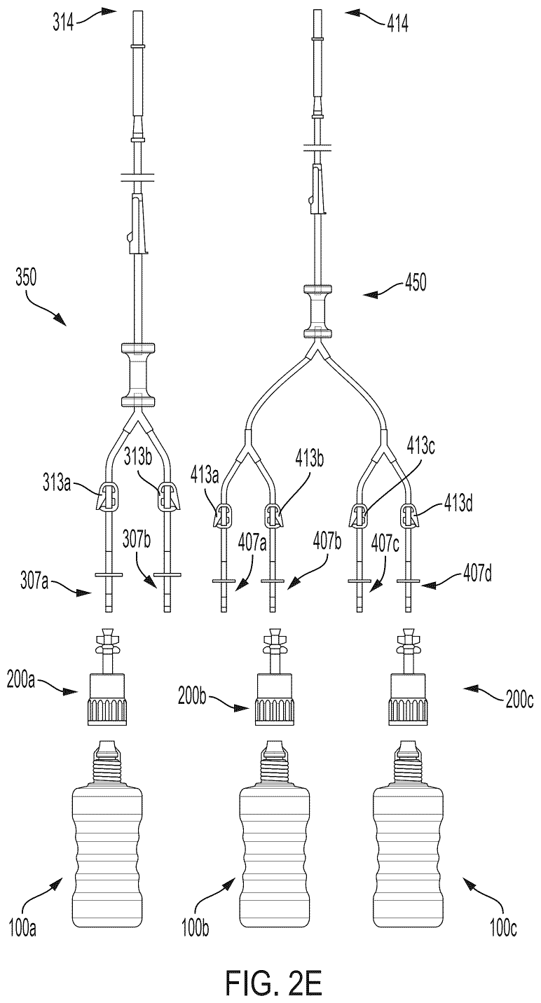

As described in further detail below, aspects of the disclosure relate to an improved lavage system, an interface apparatus for a lavage system, and lavage a method for using the system and interface. The interface described below provides several advantages such as: improving the versatility and efficiency of a lavage system; improving use safety of a lavage system; and/or eliminating or reducing the risk of contamination when using a solution with a lavage system. As one advantage, the interface apparatus described below allows a lavage system, which is typically not compatible with non-lavage or manual solution containers to be effectively used with such a container, all while improving ease of use and/or safety. FIGS. 1 and 2 show a solution container 100, which may for example be a container that is not typically compatible with and/or marketed for use with a lavage system to be used with a lavage system or apparatus 300. As described in further detail below, the interface apparatus 200 may include a threaded engagement surface for threadably engaging the interface apparatus 200 with the solution container 100. The interface apparatus 200 may include a hidden or partially covered spike interface that is configured to pierce a membrane of the solution container 100 and provide a fluid path to the lavage apparatus 300 when the threaded engagement surface is threadably engaged with corresponding threads of a solution container 100. The combination of the threaded engagement surface and partially covered spike interface improves reliability and safety of the system by ensuring that a technician or physician receives tactile feedback indicating that the interface apparatus 200 is securely connected to the solution container 100. In addition, because the spike interface is surrounded or at least partially covered by the outer walls of the interface apparatus 200, there is a decreased risk that a technician or physician may be injured by the spike interface, which may be especially critical in a hospital or other high-pressure environments.

Additional features and advantages are described with respect to the Figures in the detailed examples below.

### III. Detailed Examples

FIG. 1 shows one example of an interface apparatus 200 according to aspects of the disclosure. The interface apparatus 200 may include a gripable or tactile surface 202. As shown in FIGS. 1 and 2, the interface apparatus 200 may be configured to be engaged with a solution container 100. The solution container 100 may for example include a container interface 102 that includes a membrane or breakable seal 108, a container sealing and/or container engagement surface 106 and a threaded container engagement provision 104. In addition, the solution container 100 may for example include a housing 110 configured to contain a liquid lavage solution. The housing 110 and/or the container interface 102 may be formed of a flexible or semi-flexible plastic or other synthetic or semi-synthetic material known in the art (e.g., polyethylene, polyethylene terephthalate (PET), polystyrene, polyvinyl chloride (PVC)). In one example, the solution container 100 may be blow molded from a flexible or semi-flexible plastic or other synthetic or semi-synthetic material known in the art (e.g., polyethylene, polyethylene terephthalate (PET), polystyrene, polyvinyl chloride (PVC)). In a more specific example, the solution container 100 may be blow molded from a polypropylene.

While only one example of a solution container 100 is shown, it is noted that a solution container usable with aspects of the disclosure may for example be any known relevant solution container, which may be commercially available. As such, some elements such as the container interface 102 may vary. As described below, the interface apparatus 200 is fully contemplated to be modified and/or altered to allow for use with a variety of container and container interface types, including commercially available containers. One example of a commercially available container is sold by Becton, Dickinson and Company's as a packaged premixed PVP-I solution, sold under the name BD Surgiphor™ Sterile Wound Irrigation System.

As shown in FIGS. 1 and 2, the disclosed interface apparatus 200 is usable with a lavage apparatus 300. After the interface apparatus 200 is installed to the container 100 via the container interface 102, a fluid path may be provided to one or more fluid conduits 306 of the lavage apparatus 300. The lavage apparatus 300 may include one or more pumps (not shown) that increase the velocity and/or flow rate of the fluid provided to the lavage apparatus 300 from the container 100 via the interface apparatus 200. As shown in FIG. 2A-2D, one example of a lavage apparatus 300 may include a trigger 312, a main body 310, and a nozzle 308. The 300 may be configured to provide a pulsed or constant stream of pressurized fluid from the nozzle 308 when the trigger 312 is pressed or held down. FIGS. 2B-2D show examples of steps to connect the lavage apparatus 300 to the solution container 100 via an interface apparatus 200 according to aspects of this disclosure. The interface apparatus 200 may be installed onto the solution container 100 by threading the interface apparatus 200 thereon and tightening until the interface apparatus 200 is fully seated on the solution container 100. As shown in FIGS. 2B-2D, the lavage port 230 may have a removable cap or other sealing portion 211. Once the removeably cap or sealing portion is removed (e.g., by twisting-off), the lavage spike 307 may be installed into the lavage port 230 by pressing and/or twisting the lavage spike 307 into the lavage port 230. Once the interface apparatus 200 is installed on the solution container 100 and the lavage spike 307 is fully installed into the lavage port 230, solution from the solution container 100 may be dispensed via a lavage apparatus (e.g., lavage apparatus 300).

In one example implementation, the lavage apparatus 300 may for example be a commercially available lavage device, such as the Simpulse™ SOLO lavage device sold by Becton, Dickinson and company. In another example, the lavage apparatus 300 may be the Simpulse VariCare™ Suction Irrigator sold by Becton, Dickinson and company. Some other examples of lavage devices usable with the disclosure are the Pulsavac Plus™ and/or Pulsavac Plus ACT Wound Debridement System sold by Zimmer Biomet and/or the InterPulse™ Pulsed Lavage System sold by Stryker. While one particular example of a lavage device is shown and additional examples are provided above, it is noted that the lavage device usable with aspects of the disclosure may for example be any known relevant device, which may be commercially available. As such, some elements such as the main body 310 and/or one or more fluid conduits 306 may vary without departing from the scope of this disclosure. For example, the lavage device may have a remote or separate pump and the handpiece may have a different design from the gun-style device shown in FIG. 2A-2D.

In one example, multiple solution containers 100 and/or a single solution container 100 may be connected to a line set with multiple fluid paths allowing for the dispensing of fluid from or to at least one solution container 100 and/or another solution container, which may contain other solutions. For example, as shown in FIG. 2E, one or more solution containers (e.g., 100a, 100b, and/or 100c) may be connected to either a Y-type or “Cysto-type” tubing set used for flushing and/or irrigation (e.g., open and/or closed procedures used in cystoscopy, transurethral procedures (e.g., T.U.R), general, and arthroscopic surgical procedures). For example, one or more solution containers may be connected to a first lavage spike 307a and/or a second lavage spike 307b of a tubing set via one or more interface apparatuses 200 (e.g., 200a). A user may then dispense either a mix of fluids from the two solution containers connected to the first lavage spike 307a and/or second lavage spike 307b and/or may remove fluid from a patient into the one or more of the containers 100a, 100b, and/or 100c. The flow of fluid into or out of each container 100a, 100b, and/or 100c (and or any other containers connected to any one or combination of the first lavage spike 307a, second lavage spike 307b of the first line set 350 and/or first lavage spike 407a, second lavage spike 407b, fourth lavage spike 407c, and/or fourth lavage spike 407d of the second example line set 450) may be controlled by one or the other via a series of tubing clamps or valves 313a and/or 313b. The tubing may be in fluid communication with a catheter or other irrigation or flushing device 314 and/or 414 at a proximal end of the tubing. It is noted that while an irrigation device or flushing device is not specifically shown in FIG. 2E, any one or combination of known lavage devices including the apparatuses discussed herein, and/or any flushing or irrigation apparatus may be at a distal end of the tubing as indicated by reference 314.

In another example, one or more solution containers may be connected to a first lavage spike 407a, and/or a second lavage spike 407b, and/or a third lavage spike 407c, and/or a fourth lavage spike 407d, of a tubing set via one or more interface apparatuses 200 (e.g., 200b and/or 200c). A user may then dispense either a mix of fluids from any one, combination, or all of the containers and/or remove fluid from a patient into any one, combination, or all of the containers connected to the first lavage spike 407a, and/or second lavage spike 407b, and/or the third lavage spike 307 and/or the fourth lavage spike 407d and may control flow from or into one or the other via a series of tubing clamps or valves 313a and/or 313b. The tubing may be in fluid communication with a lavage apparatus (e.g., lavage apparatus 300 in FIGS. 2-2D), an irrigation apparatus or flushing apparatus (e.g., a catheter) at a proximal end of the tubing. It is noted that while a lavage, irrigation and/or flushing apparatus is not specifically shown, the location or connection point thereof is indicated by reference number 400 in FIG. 2E. As described below, the interface apparatus 200 is fully contemplated to be modified and/or altered to allow for use with a variety of lavage devices, irrigation devices, and/or flushing devices.

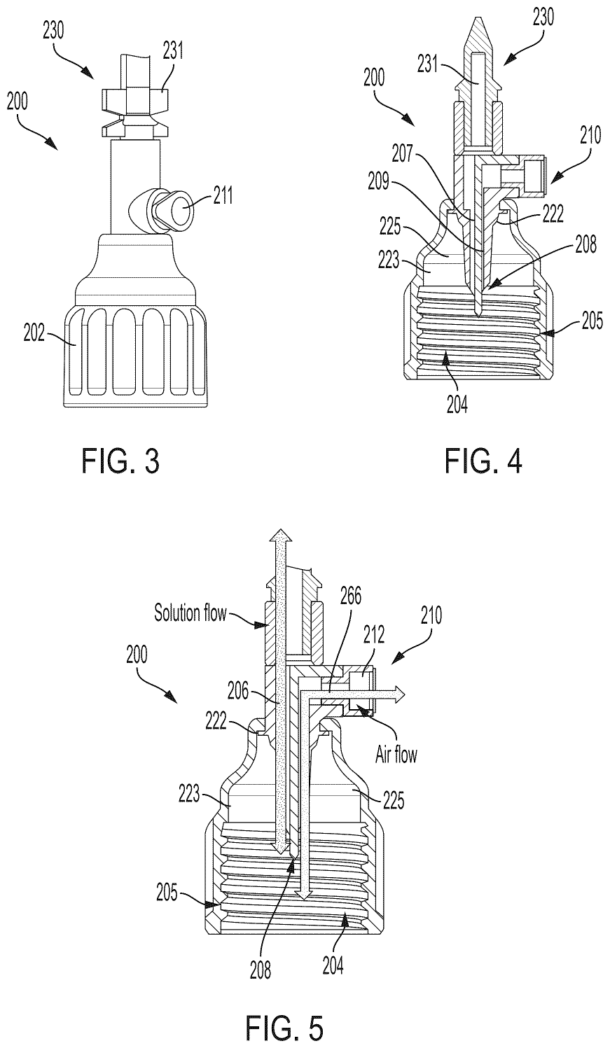

FIGS. 3-4 show examples of the interface apparatus 200 according to aspects of the disclosure. The interface apparatus 200 may for example include, a gripable or tactile surface 202. The tactile surface 202 may for example comprise a series of radially spaced concavities that allow a user to easily grip the interface apparatus 200 even when wearing gloves. In addition, the tactile surface 202 may for example provide extra grip when technician or physicians gloves are wet or otherwise covered in material that may reduce the coefficient of friction between the users gloves and the interface apparatus 200. It is noted that while a series of concavities or troughs are shown in FIG. 1, any surface irregularities or patterns, that can improve a user's grip on the interface apparatus 200 may be provided. Some example alternatives include a ribs, grooves, dots, and/or a roughened surface.

The interface apparatus 200 may further include a container interface receiving portion 205. The container receiving portion 205 may include an inner radial wall with threads 204 that are configured to threadably engage with the threaded container engagement provisions 104 of solution container 100 (FIG. 1). In some examples, the interface receiving portion 205 of the interface apparatus 200 may include a child-lock type or an anti-open lock within the threads. For example, anti-open lock may require a second action by a technician or user other than a twisting motion. For example, the second action may be the application of a squeezing or and/or the pressing of a button or a plurality of buttons which combined with a twisting motion would then engage the threads of the solution container 100 and allow for removal. In the aforementioned example, if the second action is not performed, the interface apparatus 200 may simply turn on the solution container (i.e., without engagement of the threads 204) and/or may be locked from turning. This featured may prevent accidental or unintentional removal of the interface apparatus 200 from the solution container 100.

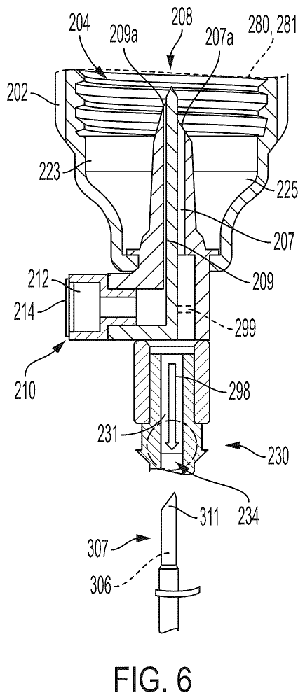

As shown in the example cross-section view of the interface apparatus 200 in FIGS. 4-6, container interface receiving portion 205 may have a spike interface 208 therein. The spike interface 208 may for example extend from an inner wall 222 of the interface apparatus 200. The spike interface 208 may taper down to a reduced cross-section point and/or may have a sharpened point (e.g., as shown in FIGS. 4 and 6). The point or sharpened point may for be dimensioned or otherwise configured to pierce a membrane or a pierceable section of a solution container when the interface apparatus 200 is threaded onto or otherwise installed onto the solution container (e.g., as shown in FIG. 2A-2D). The spike interface 208 may include one or more fluid conduits configured to provide fluid communication with the solution container 100 when the interface apparatus 200 is installed onto the container interface 102 of the solution container 100. The spike interface 208 may include a fluid passage 207 to provide fluid (e.g., a lavage solution contained within the solution container) supply to the one or more fluid conduits 306 of the lavage apparatus 300. The arrow 206 in FIG. 5 represents one example fluid path of the lavage solution from the container 100 (FIG. 1), through the interface apparatus 200, to be supplied to the lavage apparatus 300 (FIG. 2A-2D).

The spike interface 208 may further include a vent passage 209. The vent passage may for example provide air or gas to the solution container 100 to equalize the pressure within the solution container 100 as the lavage solution is removed from the container 100 via the fluid passage 207. In one example implementation, the vent passage may for example include a vent 210 with a 1-way valve, or membrane, such as a hydrophobic membrane 212. In one example implementation, the hydrophobic membrane 212 prevents liquid solution from passing therethrough while allowing air to freely move into and/or out of the solution container 100 and thus allowing the pressure withing the solution container to equalize as the solution is removed from the solution container 100 by the lavage device. In some examples, the vent passage may for example have a filter or other membrane, which may be a sterile filter or membrane at the opening of the hydrophobic membrane to prevent contaminants from entering the solution container. The arrow 266 in FIG. 5 shows one example of the fluid air/gas flow through the vent passage 209 and the hydrophobic membrane 212 (e.g., when the interface apparatus 200 is installed onto the solution container 100).

The interface apparatus 200 may further include an end surface 223, the end surface 223 may for example provide a surface transition from the threads 204 of the interface apparatus 200 to a non-threaded radial surface and/or a blind wall 225. The end surface 223 and/or blind wall may provide a mechanical stopping point when the interface apparatus 200 is threadingly engaged with the solution container 100. The mechanical stopping point provided by the end surface 223 and/or the blind wall 225 provide the additional advantage of indicating a user that the interface apparatus 200 is properly installed onto the solution container 100 and when the spike interface 208 has fully pierced the membrane or a pierceable section of a solution container 100. In some examples, the end surface 223 may for example include an additional feedback mechanism or device. For example, the blind wall may have an elastic or semi-elastic secondary wall or other protrusion that provides a tactile or audible “click” when the interface apparatus 200 is fully engaged with the solution container 100.

The engagement of any one or a combination of the threads 204, the spike interface 208, and/or blind wall 225 or end surface 223 may create a seal between the solution container 100 and the interface apparatus 200 to ensure that a lavage solution does not leak from the interface and/or the container. For example, to install the interface apparatus 200 onto the solution container, a user may align the receiving portion 205 with the container interface 102 of the solution container 100 and while gripping the solution container 100 and the gripable or tactile surface 202 of the interface apparatus 200, press and twist the interface apparatus 200 onto the container interface 102. Once the threaded container engagement provisions 104 and the receiving threads 204 are threadably engaged, the user may continue to twist the interface apparatus with respect to the solution container 100 as the spike interface 208 punctures a membrane or pierceable portion 108 of the solution container 100—and continue to twist the interface until the resistance is felt as the container interface 102 bottoms out against the end surface 223 and/or blind wall 225 of the interface apparatus 200. The increased resistance felt by the user as the container interface 102 bottoms out against the end surface 223 and/or blind wall 225 of the interface apparatus 200 provides an indication that the interface apparatus 200 is fully installed and that a seal has been established with the solution container 100.

As an alternative to the aforementioned aspect, a user may align the spike interface 208 with the membrane or pierceable portion 108 of the solution container and use the spike interface 208 to puncture the membrane or pierceable portion 108 before the threaded container engagement provisions 104 and the receiving threads 204 are threadably engaged. After the membrane or pierceable portion 108 is punctured by the spike interface 208, the user may engage the threaded container engagement provisions 104 and the receiving threads 204 and twist the interface apparatus 200 with respect to the solution container 100 until resistance is felt as the container interface 102 bottoms out against the end surface 223 and/or blind wall 225 of the interface apparatus 200. Similarly to the aspects above, the increased resistance felt by the user as the container interface 102 bottoms out against the end surface 223 and/or blind wall 225 of the interface apparatus 200 provides an indication that the interface apparatus 200 is fully installed and that a seal has been established with the solution container 100.

In one example usable with the aforementioned examples, the interface apparatus 200 may for example include a protection seal 280. The protection seal may further prevent contamination of the spike interface 208 and/or may protect a user from potentially being injured by unintentionally contacting the spike interface 208. In one example, the 280 may be a polymeric film or foil that is either removable prior to the interface apparatus 200 being installed onto the solution container 100 and/or may be configured to break or perforate as the user is installing the interface apparatus 200 onto the solution container 100 using the method described above. In another example, the spike interface 208 may be covered in a spike sheath (not shown) until time of activation or use. As another alternative, the interface apparatus 200 may incorporate a retractable spike interface 208 that is only exposed as the interface apparatus 200 is being threaded onto the solution container 100. In the aforementioned example, if the interface apparatus 200 is not in use, the spike interface 208 remains hidden and the spike interface 208 is only exposed when the interface apparatus 200 is threaded onto the container interface 102 of the solution container 100. In the aforementioned example, the interface apparatus 200 may include a split polymeric material 281 that covers the spike interface 208, and as the interface apparatus 200 is threaded onto the container interface 102 of the solution container 100, the polymeric material splits to expose the spike interface 208. The aforementioned split polymeric material may be an elastic material that is configured to retain its original shape and thus is configured to again cover the spike interface 208 when the interface apparatus 200 is removed from the container interface 102. In one example, the split polymeric material 281 may for example be silicon or any other material that provides the required flexibility to allow spike interface 208 to penetrate the material while still maintaining or reverting back to its original shape. In one example, the split polymeric material 281 may for example be formed of a semi-rigid silicone or the like. The aforementioned example provides improved safety to a user both before the interface apparatus 200 is installed and after the interface apparatus 200 is removed.

Either the protection seal 280 or the split polymeric material 281 may for example be coated or treated with, and/or may contain an antimicrobial agent, which may further reduce the potential of contamination of the solution or the solution path and help maintain sterility of the lavage solution.

As shown in FIG. 6, in one example aspect of the disclosure, the interface apparatus 200 may include a check valve or one way valve 299. The check valve or one way valve could include any known valve, membrane or combination thereof. The check valve 299 may for example only allow flow in the direction of arrow 298, which prevents potential contaminants either in the lavage apparatus 300 and or the one or more fluid conduits 306 from being pulled back into the solution container 100 due to any pressure differences at the pump of the lavage apparatus 300 and the solution container 100. With the check valve 299 incorporated into the interface apparatus 200, risk of contamination of the lavage solution is further reduced and/or completely eliminated.

As shown in FIG. 6, the interface apparatus 200 may further include a lavage port 230. The lavage port 230 may for example have a channel or fluid path 231 that is in fluid communication with the fluid passage 207. As shown in FIG. 3, the fluid passage may for example have a lavage port seal 234. The lavage port seal 234 may for example be a foil or film seal and/or may be molded as part of the interface apparatus 200 and may be configured to break or otherwise have an opening created therein by a sharpened tip 311 of a lavage spike 307 of the lavage device (e.g., lavage apparatus 300 in FIG. 2A-2D). The inner diameter of the channel or fluid path 231 may for example be dimensioned to be slightly smaller than or equal to at least a portion of the lavage spike in dimension, thus creating a sealing fit with the lavage spike 307. In addition, the lavage port 230 and/or the area surrounding the channel or fluid path 231 may for example be formed of a flexible or semi-flexible material. Any one or a combination of the aforementioned features may allow for a liquid impervious seal to be formed between the interface apparatus 200 and the lavage spike 307 when the lavage spike 307 is pressed into the lavage port 230. In preparing the lavage apparatus 300 for use, the user may press the lavage spike 307 into the lavage port 230, thus causing the lavage port seal 234 to break or otherwise open a fluid path between the lavage spike 307 and the fluid passage 207 of the interface apparatus 200. Thus, when the lavage spike 307 is installed into the interface apparatus 200, and the solution container 100 is connected to the interface apparatus 200, a lavage solution can be pumped from the solution container 100 to the lavage apparatus 300.

In addition to preventing contamination of the interface apparatus 200, the lavage port seal 234 further provides improvement in useability, efficiency, and may reduce waste of lavage fluid or accidents. Typically, technicians and healthcare practitioners are accustomed to hanging fluids on an IV pole and then installing the lavage spike 307 of the lavage apparatus 300 directly into a bag of containing a solution. Thus, if no lavage port seal 234 was present, and the technician were to hang the solution container 100 with the interface apparatus 200 already installed thereon on an IV pole, the lavage fluid would pour out of the solution container 100 through the channel or fluid path 231. The lavage port seal 234 allows a technician to use a solution container 100 with the interface apparatus 200 without having to alter the typical workflow that they may be accustomed to. Additionally, the lavage port 230 may contain features, markings, or engravings that direct the user to insert the lavage spike 307 fully into the lavage port 230 to prevent the lavage spike from falling out. The markings/engravings may visually indicate to the user that the lavage spike is properly inserted when it aligns to the engravings i.e. “spike until line”.

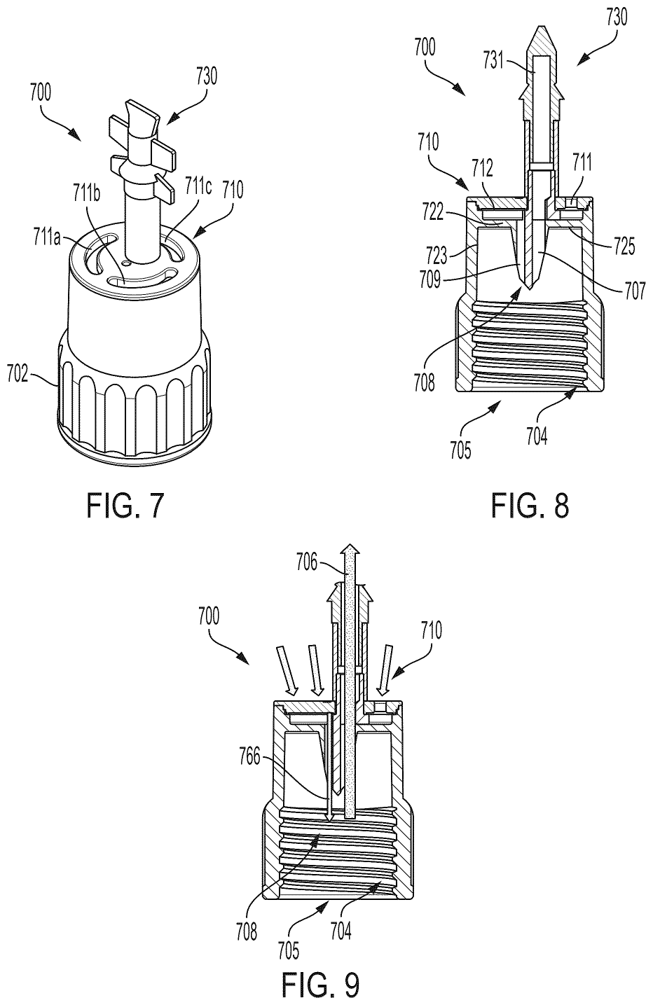

FIGS. 7-10 show examples of the interface apparatus 700 according to aspects of the disclosure. In some examples, the interface apparatus 700 described below may share features with, may include features that replace features that are described above, and/or may be analogous with the interface apparatus 200 described above. In addition, aspects described throughout this disclosure are usable with the features described below with respect to FIGS. 7-10.

The interface apparatus 400 may for example include, a gripable or tactile surface 702. The tactile surface 702 may for example comprise a series of radially spaced concavities that allow a user to easily grip the interface apparatus 700 even when wearing gloves. In addition, the tactile surface 702 may for example provide extra grip when technician or physicians gloves are wet or otherwise covered in material that may reduce the coefficient of friction between the users gloves and the interface apparatus 700. It is noted that while a series of concavities or troughs are shown in FIG. 7, any surface irregularities or patterns that can improve a user's grip on the interface apparatus 700 may be provided. Some example alternatives include a ribs, grooves, dots, and/or a roughened surface.

The interface apparatus 700 may further include a container interface receiving portion 705. The container receiving portion 705 may include an inner radial wall with threads 704 that are configured to threadably engage with the threaded container engagement provisions 104 of solution container 100 (FIGS. 1, 2A-2E). In some examples, the interface receiving portion 705 of the interface apparatus 700 may include an child-lock type or an anti-open lock within the threads. For example, anti-open lock may require a second action by a technician or user other than a twisting motion. For example, the second action may be the application of a squeezing or and/or the pressing of a button or a plurality of buttons which combined with a twisting motion would then engage the threads of the solution container 100 and allow for removal. In the aforementioned example, if the second action nis not performed, the interface apparatus 700 may simply turn on the solution container (i.e., without engagement of the threads 704) and/or may be locked from turning. This featured may prevent accidental or unintentional removal of the interface apparatus 700 from the solution container 100.

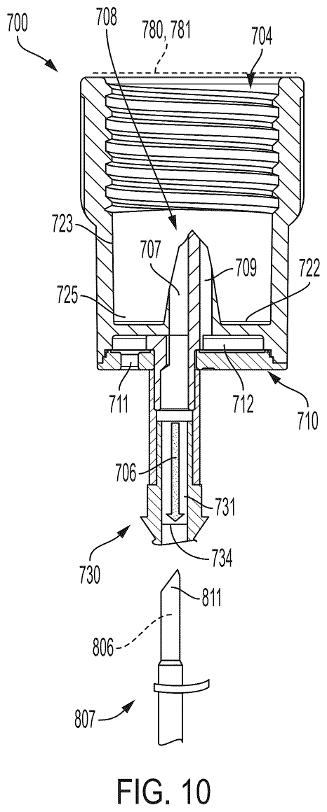

As shown in the example cross-section view of the interface apparatus 700 in FIGS. 8-10, container interface receiving portion 705 may have a spike interface 708 therein. The spike interface 708 may for example extend from an inner wall 722 of the interface apparatus 700. The spike interface 708 may taper down to a reduced cross-section point and/or may have a sharpened point (e.g., as shown in FIGS. 8-10). The point or sharpened point may for be dimensioned or otherwise configured to pierce a membrane or a pierceable section of a solution container when the interface apparatus 700 is threaded onto or otherwise installed onto the solution container (e.g., as shown in FIG. 2A-2D). The spike interface 708 may include one or more fluid conduits configured to provide fluid communication with the solution container 100 when the interface apparatus 700 is installed onto the container interface 102 (FIG. 1) of the solution container 100. The spike interface 708 may include a fluid passage 707 to provide fluid (e.g., a lavage solution contained within the solution container) supply to the one or more fluid conduits 308 of the lavage apparatus (e.g., apparatus 300 in FIGS. 2A-2D). The arrow 706 in FIGS. 9 and 10 represent one example fluid path of the lavage solution from the container 100 (FIGS. 1-2E), through the interface apparatus 200, to be supplied to the lavage apparatus 300, 314, 415 (FIG. 2A-2E).

The spike interface 708 may further include a vent passage 709. The vent passage may for example provide air or gas to the solution container 100 to equalize the pressure within the solution container 100 as the lavage solution is removed from the container 100 via the fluid passage 707. In one example implementation, the vent passage may for example include a plurality of vents 711 with a 1-way valve, or membrane, such as a hydrophobic membrane 712 (FIG. 10). As shown in the example of FIG. 7, the plurality of vents 711 may for example include a first vent 711a, a second vent 711b, and/or a third vent 711c, which may be semi-circular and follow the outline of the top surface of the interface apparatus 700. It is noted that while three vent openings are shown in FIG. 7, and number of shape of openings may be provided without departing from the scope of this disclosure. In one example implementation, the hydrophobic membrane 712 prevents liquid solution from passing therethrough while allowing air to freely move into and/or out of the solution container 100 and thus allowing the pressure withing the solution container to equalize as the solution is removed from the solution container 100 by the lavage device. In some examples, the vent passage may for example have a filter or other membrane, which may be a sterile filter or membrane at the opening of the hydrophobic membrane to prevent contaminants from entering the solution container. The arrows 710 and 766 in FIG. 9 shows one example of the fluid air/gas flow through the vent passage 709 and the hydrophobic membrane 712 (e.g., when the interface apparatus 700 is installed onto the solution container 100).

The interface apparatus 700 may further include an end surface 723, the end surface 223 may for example provide a surface transition from the threads 204 of the interface apparatus 200 to a non-threaded radial surface and/or a blind wall 225. The end surface 223 and/or blind wall may provide a mechanical stopping point when the interface apparatus 200 is threadingly engaged with the solution container 100. The mechanical stopping point provided by the end surface 223 and/or the blind wall 725 provide the additional advantage of indicating a user that the interface apparatus 700 is properly installed onto the solution container 100 and when the spike interface 708 has fully pierced the membrane or a pierceable section of a solution container 100. In some examples, the end surface 723 may for example include an additional feedback mechanism or device. For example, the blind wall may have an elastic or semi-elastic secondary wall or other protrusion (not shown) that provides a tactile or audible “click” when the interface apparatus 700 is fully engaged with the solution container 100.

The engagement of any one or a combination of the threads 704, the spike interface 708, and/or blind wall 725 or end surface 723 may create a seal between the solution container 100 and the interface apparatus 700 to ensure that a lavage solution does not leak from the interface and/or the container. For example, to install the interface apparatus 200 onto the solution container, a user may align the receiving portion 705 with the container interface 102 of the solution container 100 and while gripping the solution container 100 and the gripable or tactile surface 702 of the interface apparatus 700, press and twist the interface apparatus 700 onto the container interface 102. Once the threaded container engagement provisions 104 and the receiving threads 704 are threadably engaged, the user may continue to twist the interface apparatus with respect to the solution container 100 as the spike interface 708 punctures a membrane or pierceable portion 108 of the solution container 100—and continue to twist the interface until the resistance is felt as the container interface 102 bottoms out against the end surface 723 and/or blind wall 725 of the interface apparatus 700. The increased resistance felt by the user as the container interface 102 bottoms out against the end surface 723 and/or blind wall 725 of the interface apparatus 700 provides an indication that the interface apparatus 700 is fully installed and that a seal has been established with the solution container 100.

As an alternative to the aforementioned aspect, a user may align the spike interface 708 with the membrane or pierceable portion 108 of the solution container and use the spike interface 708 to puncture the membrane or pierceable portion 108 before the threaded container engagement provisions 104 and the receiving threads 704 are threadably engaged. After the membrane or pierceable portion 108 is punctured by the spike interface 708, the user may engage the threaded container engagement provisions 104 and the receiving threads 704 and twist the interface apparatus 700 with respect to the solution container 100 until resistance is felt as the container interface 102 bottoms out against the end surface 723 and/or blind wall 725 of the interface apparatus 700. Similarly to the aspects above, the increased resistance felt by the user as the container interface 102 bottoms out against the end surface 723 and/or blind wall 725 of the interface apparatus 700 provides an indication that the interface apparatus 700 is fully installed and that a seal has been established with the solution container 100.

In one example usable with the aforementioned examples, and similar to the aspects described above with respect to the interface apparatus 200, the interface apparatus 700 may for example include a protection seal 780. The protection seal may further prevent contamination of the spike interface 708 and/or may protect a user from potentially being injured by unintentionally contacting the spike interface 708. In one example, the 780 may be a polymeric film or foil that is either removable prior to the interface apparatus 700 being installed onto the solution container 100 and/or may be configured to break or perforate as the user is installing the interface apparatus 700 onto the solution container 100 using the method described above. In another example, the spike interface 708 may be covered in a spike sheath (not shown) until time of activation or use. As another alternative, the interface apparatus 700 may incorporate a retractable spike interface 208 that is only exposed as the interface apparatus 700 is being threaded onto the solution container 100. In the aforementioned example, if the interface apparatus 700 is not in use, the spike interface 708 remains hidden and the spike interface 708 is only exposed when the interface apparatus 700 is threaded onto the container interface 102 of the solution container 100. In the aforementioned example, the interface apparatus 700 may include a split polymeric material 781 that covers the spike interface 708, and as the interface apparatus 700 is threaded onto the container interface 102 of the solution container 100, the polymeric material splits to expose the spike interface 708. The aforementioned split polymeric material may be an elastic material that is configured to retain its original shape and thus is configured to again cover the spike interface 708 when the interface apparatus 700 is removed from the container interface 102. In one example, the split polymeric material 781 may for example be silicon or any other material that provides the required flexibility to allow spike interface 708 to penetrate the material while still maintaining or reverting back to its original shape. In one example, the split polymeric material 781 may for example be formed of a semi-rigid silicone or the like. The aforementioned example provides improved safety to a user both before the interface apparatus 700 is installed and after the interface apparatus 700 is removed.

Either the protection seal 780 or the split polymeric material 781 may for example be coated or treated with, and/or may contain an antimicrobial agent, which may further reduce the potential of contamination of the solution or the solution path and help maintain sterility of the lavage solution.

While not shown in FIGS. 7-10, in one example aspect of the disclosure, the interface apparatus 700 may include a check valve or one way valve (e.g., similar to one way valve 299 in FIG. 6). The check valve or one way valve could include any known valve, membrane or combination thereof. The check valve may for example only allow flow in the direction of arrow 706, which prevents potential contaminants either in the lavage apparatus 300 and or the one or more fluid conduits (e.g., 306, 350, and/or 450, in FIGS. 2A and 2E) from being pulled back into the solution container 100 due to any pressure differences at the pump of the lavage apparatus 300 and the solution container 100. With the check valve incorporated into the interface apparatus 700, risk of contamination of the lavage solution is further reduced and/or completely eliminated.

As shown in FIG. 10, the interface apparatus 700 may further include a lavage port 730. The lavage port 730 may for example have a channel or fluid path 731 that is in fluid communication with the fluid passage 707. As shown in FIG. 10, the fluid passage may for example have a lavage port seal 734. The lavage port seal 734 may for example be a foil or film seal and/or may be molded as part of the interface apparatus 700 and may be configured to break or otherwise have an opening created therein by a sharpened tip 811 of a lavage spike 807 of the lavage device (e.g., lavage apparatus 300 and/or 315 or 415 in FIG. 2A-2E). The inner diameter of the channel or fluid path 731 may for example be dimensioned to be slightly smaller than or equal to at least a portion of the lavage spike in dimension, thus creating a sealing fit with the lavage spike 807. In addition, the lavage port 730 and/or the area surrounding the channel or fluid path 731 may for example be formed of a flexible or semi-flexible material. Any one or a combination of the aforementioned features may allow for a liquid impervious seal to be formed between the interface apparatus 700 and the lavage spike 807 when the lavage spike 807 is pressed into the lavage port 730. In preparing the lavage apparatus 300 for use, the user may press the lavage spike 807 into the lavage port 730, thus causing the lavage port seal 734 to break or otherwise open a fluid path between the lavage spike 807 and the fluid passage 707 of the interface apparatus 700. Thus, when the lavage spike 807 is installed into the interface apparatus 700, and the solution container 100 is connected to the interface apparatus 800, a lavage solution can be pumped from the solution container 100 to the lavage apparatus (e.g., 300 and/or 315, or 415).

In addition to preventing contamination of the interface apparatus 700, the lavage port seal 734 further provides improvement in useability, efficiency, and may reduce waste of lavage fluid or accidents. Typically, technicians and healthcare practitioners are accustomed to hanging fluids on an IV pole and then installing the lavage spike 807 of the lavage apparatus 800 directly into a bag of containing a solution. Thus, if no lavage port seal 734 was present, and the technician were to hang the solution container 100 with the interface apparatus 700 already installed thereon on an IV pole, the lavage fluid would pour out of the solution container 100 through the channel or fluid path 731. The lavage port seal 734 allows a technician to use a solution container 100 with the interface apparatus 700 without having to alter the typical workflow that they may be accustomed to.

Additionally, the lavage port 730 may contain features, markings, or engravings that direct the user to insert the lavage spike 807 fully into the lavage port 730 to prevent the lavage spike from falling out. The markings/engravings may visually indicate to the user that the lavage spike is properly inserted when it aligns to the engravings i.e. “spike until line.

This written description uses examples to disclose aspects of the invention, including the preferred embodiments, and also to enable any person skilled in the art to practice the aspects thereof, including making and using any devices or systems and performing any incorporated methods. The patentable scope of these aspects is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. Aspects from the various embodiments described, as well as other known equivalents for each such aspect, can be mixed and matched by one of ordinary skill in the art to construct additional embodiments and techniques in accordance with principles of this application.

### IV. Example Implementations

The following clauses include example implementations of the aspects described herein.

Clause 1. An interface apparatus usable with a solution container and an irrigation apparatus, the interface apparatus comprising: a receiving portion configured to provide a sealed connection with a container interface of the solution container, wherein the receiving portion further comprises a spike interface therein; and the spike interface includes a first fluid path for providing fluid communication between the solution container and the irrigation apparatus via the interface apparatus.

Clause 2. The interface apparatus of clause 1, wherein the spike interface has a tip configured to pierce a portion of the solution container, wherein the first fluid path is configured to fluidly communicate with the solution container when the solution container is pierced.

Clause 3. The interface apparatus of any of the above clauses, wherein the spike interface includes a second fluid path, wherein the second fluid path is configured to provide the solution container with fluid communication to an atmosphere outside the solution container via a vent when the solution container is pierced by the spike interface.

Clause 4. The interface apparatus of any of the above clauses, wherein the vent further comprises at least one of a one-way valve, a membrane, or a sterile filter.

Clause 5. The interface apparatus of any of the above clauses, wherein the interface apparatus is configured to be installed onto the container interface of the solution container.

Clause 6. The interface apparatus of any of the above clauses, wherein the receiving portion further comprises engagement threads that are configured to threadingly engage with threaded container engagement provisions of the container interface, wherein the threading engagement between the engagement threads and the threaded container engagement provisions causes the spike interface to pierce the solution container at the container interface as the interface apparatus is rotated with respect to the solution container.

Clause 7. The interface apparatus of any of the above clauses, further comprising an irrigation port configured interface with and provide fluid communication from the solution container to an irrigation apparatus connector.

Clause 8. The interface apparatus of any of the above clauses, wherein the irrigation apparatus connector is an irrigation device spike, and wherein the irrigation apparatus connector is configured to provide the fluid communication from the solution container to the irrigation apparatus connector and irrigation apparatus when the irrigation device spike is pressed into the lavage port.

Clause 9. The interface apparatus of any of the above clauses, wherein the irrigation port further comprises an irrigation port seal, wherein the irrigation port seal is configured to be pierced by the irrigation device spike when the irrigation device spike is pressed into the irrigation port.

Clause 10. A lavage device and interface device, the lavage device comprising a nozzle and a pump in fluid communication with the nozzle, wherein the pump provides a pressurized stream of lavage solution supplied from a solution container, wherein the interface device comprises: a receiving portion configured to provide a sealed connection with a container interface of the solution container, wherein the receiving portion further comprises a spike interface therein; and the spike interface includes a first fluid path for providing fluid communication between the solution container and the lavage device via the interface apparatus.

Clause 11. The lavage device and interface device of clause 10, wherein the spike interface has a tip configured to pierce a portion of the solution container, wherein the first fluid path is configured to fluidly communicate with the solution container when the solution container is pierced.

Clause 12. The lavage device and interface device of any of the above clauses, wherein the spike interface includes a second fluid path, wherein the second fluid path is configured to provide the solution container with fluid communication to an atmosphere outside the solution container via a vent when the solution container is pierced by the spike interface.

Clause 13. The lavage device and interface device of any of the above clauses, wherein the vent further comprises at least one of a one-way valve, a membrane, or a sterile filter.

Clause 14. The lavage device and interface device of any of the above clauses, wherein the interface apparatus is configured to be installed onto the container interface of the solution container.

Clause 15. The lavage device and interface device of any of the above clauses, wherein the receiving portion further comprises engagement threads that are configured to threadingly engage with threaded container engagement provisions of the container interface, wherein the threading engagement between the engagement threads and the threaded container engagement provisions causes the spike interface to pierce the solution container at the container interface as the interface apparatus is rotated with respect to the solution container.

Clause 16. The lavage device and interface device of any of the above clauses, further comprising a lavage port configured interface with and provide fluid communication from the solution container to a lavage apparatus connector.

Clause 17. The lavage device and interface device of any of the above clauses, wherein the lavage apparatus connector is a lavage spike, and wherein the lavage apparatus connector is configured to provide the fluid communication from the solution container to the lavage apparatus connector and lavage apparatus when the lavage spike is pressed into the lavage port.

Clause 18. The lavage device and interface device of any of the above clauses, wherein the lavage port further comprises a lavage port seal, wherein the lavage port seal is configured to be pierced by the lavage spike when the lavage spike is pressed into the lavage port.

Clause 19. A method for providing a fluid path from a solution container to an irrigation device, the method comprising: connecting a receiving portion of an interface apparatus to the solution container by puncturing a portion of the solution container with a spike interface of the interface apparatus, wherein puncturing a portion of the solution container with the spike interface establishes a fluid connection between the solution container and the irrigation apparatus via the interface apparatus.

Clause 20. The method of clause 19, wherein the receiving portion further comprises engagement threads and the solution container further comprises engagement provisions, and wherein the method further comprises threadingly engaging the engagement threads of the receiving portion with the threaded container engagement provisions to provide a sealed interface between the interface apparatus and the solution container.

1. An interface apparatus usable with a solution container and an irrigation apparatus, the interface apparatus comprising:
a receiving portion configured to provide a sealed connection with a container interface of the solution container, wherein the receiving portion further comprises a spike interface therein; and
the spike interface includes a first fluid path for providing fluid communication between the solution container and the irrigation apparatus via the interface apparatus,
wherein the receiving portion comprises engagement threads that are configured to provide threading engagement with threaded container engagement provisions of the container interface, and
wherein the threading engagement causes the spike interface to pierce the solution container at the container interface as the interface apparatus is rotated with respect to the solution container.

2. The interface apparatus of claim 1, wherein the spike interface has a tip configured to pierce a portion of the solution container, wherein the first fluid path is configured to fluidly communicate with the solution container when the solution container is pierced.

3. The interface apparatus of claim 2, wherein the spike interface includes a second fluid path, wherein the second fluid path is configured to provide the solution container with fluid communication to an atmosphere outside the solution container via a vent when the solution container is pierced by the spike interface.

4. The interface apparatus of claim 3, wherein the vent further comprises at least one of a one-way valve, a membrane, or a sterile filter.

5. The interface apparatus of claim 2, wherein the interface apparatus is configured to be installed onto the container interface of the solution container.

6. (canceled)

7. The interface apparatus of claim 1, further comprising an irrigation port configured interface with and provide fluid communication from the solution container to an irrigation apparatus connector.

8. The interface apparatus of claim 7, wherein the irrigation apparatus connector is an irrigation device spike, and wherein the irrigation apparatus connector is configured to provide the fluid communication from the solution container to the irrigation apparatus connector and irrigation apparatus when the irrigation device spike is pressed into the irrigation port.

9. The interface apparatus of claim 8, wherein the irrigation port further comprises an irrigation port seal, wherein the irrigation port seal is configured to be pierced by the irrigation device spike when the irrigation device spike is pressed into the irrigation port.

10. A lavage device and interface device, the lavage device comprising a nozzle and a pump in fluid communication with the nozzle, wherein the pump provides a pressurized stream of lavage solution supplied from a solution container, wherein the interface device comprises:
a receiving portion configured to provide a sealed connection with a container interface of the solution container, wherein the receiving portion further comprises a spike interface therein; and
the spike interface includes a first fluid path for providing fluid communication between the solution container and the lavage device via the interface device,
wherein the receiving portion further comprises engagement threads that are configured to provide threading engagement with threaded container engagement provisions of the container interface, and
wherein the threading engagement causes the spike interface to pierce the solution container at the container interface as the interface apparatus is rotated with respect to the solution container.

11. The lavage device and interface device of claim 10, wherein the spike interface has a tip configured to pierce a portion of the solution container, wherein the first fluid path is configured to fluidly communicate with the solution container when the solution container is pierced.

12. The lavage device and interface device of claim 11, wherein the spike interface includes a second fluid path, wherein the second fluid path is configured to provide the solution container with fluid communication to an atmosphere outside the solution container via a vent when the solution container is pierced by the spike interface.

13. The lavage device and interface device of claim 12, wherein the vent further comprises at least one of a one-way valve, a membrane, or a sterile filter.

14. The lavage device and interface device of claim 11, wherein the interface apparatus is configured to be installed onto the container interface of the solution container.

15. (canceled)

16. The lavage device and interface device of claim 10, further comprising a lavage port configured interface with and provide fluid communication from the solution container to a lavage apparatus connector.

17. The lavage device and interface device of claim 16, wherein the lavage apparatus connector is a lavage spike, and wherein the lavage apparatus connector is configured to provide the fluid communication from the solution container to the lavage apparatus connector and lavage apparatus when the lavage spike is pressed into the lavage port.

18. The lavage device and interface device of claim 17, wherein the lavage port further comprises a lavage port seal, wherein the lavage port seal is configured to be pierced by the lavage spike when the lavage spike is pressed into the lavage port.

19. A method for providing a fluid path from a solution container to an irrigation device, the method comprising:
connecting a receiving portion of an interface apparatus to the solution container by puncturing a portion of the solution container with a spike interface of the interface apparatus,
wherein puncturing a portion of the solution container with the spike interface establishes a fluid connection between the solution container and the irrigation apparatus via the interface apparatus,
wherein the receiving portion further comprises engagement threads and the solution container further comprises engagement provisions, and
wherein the method further comprises threadingly engaging the engagement threads of the receiving portion with the engagement provisions to provide a sealed interface between the interface apparatus and the solution container.

20. (canceled)
